(19) United States
(12) Patent Application Publication
(10) Pub. No.: US 2026/0132198 A1
(43) Pub. Date: May 14, 2026

(54) SELF-REGULATINGCHIMERIC ANTIGEN RECEPTOR FOR NATURAL KILLER CELLS

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Meisam Naeimi KARAROUDI, Columbus, OH (US)

(21) Appl. No.: 19/118,991

(22) PCT Filed: Oct. 6, 2023
(86) PCT No.: PCT/US2023/034625

§ 371 Date: Apr. 7, 2025

Related U.S. Application Data
(60) Provisional application No. 63/414,451, filed on Oct. 7, 2022.

(51) Int. Cl. C07K 16/28; A61K 40/15; A61K 40/31; A61K 40/42; C12N 5/0783; C12N 9/22; C12N 15/11; C12N 15/86; C12N 15/90

(52) U.S. Cl. CPC C07K 16/2803; A61K 40/15; A61K 40/31; A61K 40/421; C12N 5/0646; C12N 9/226; C12N 15/11; C12N 15/86; C12N 15/907; C12N 2310/20; C12N 2510/00; C12N 2750/14143

(57) ABSTRACT

Disclosed are self-regulated chimeric antigen receptors and methods of making the same. Also, disclosed herein are methods of treating a cancer through the administration of NK cells and/or NK T cells comprising said chimeric antigen receptors.

### II. CROSS REFERENCE TO RELATED APPLICATIONS

This application claims the benefit of U.S. Provisional Application No. 63/414,451, filed on Oct. 7, 2022 which is incorporated herein by reference in its entirety.

### I. STATEMENT OF GOVERNMENT SUPPORT

This invention was made with government support under Grant No. CA232561 awarded by the National Institutes of Health. The government has certain rights in the invention.

### III. BACKGROUND

Human peripheral blood natural killer (NK) cells have intense antitumor activity and have been used successfully in several clinical trials. Modifying NK cells with a chimeric antigen receptor (CAR) can improve their targeting and increase specificity. However, NK cells can be susceptible to regulatory suppression by several checkpoint inhibitors as well as by suppressor of cytokine signaling 3 (SOCS3) which negatively regulates cytokine signaling through the JAK/STAT pathway. What are needed are new ways to modify NK cells and/or NK T cells for the production of a better immunotherapy.

### IV. SUMMARY

Disclosed are methods and compositions related to self-regulating CAR expression in Natural Killer (NK) cells and Natural Killer (NK) T cells.

In one aspect, disclosed herein are chimeric antigen receptors (CARs) comprising an endodomain comprising a STAT3 phosphorylation site (such as, for example, an IL-6, IFN-λR1, IL-2R, IL-10, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33). In one aspect, the CAR can further comprise a co-stimulatory domain (such as, for example, a 2B4 domain, a CD28 co-stimulatory domain, a CD32 domain, a 4-1BB co-stimulatory domain, or any combination thereof).

Also disclosed herein are plasmids encoding the CAR of any preceding aspect.

In one aspect, also disclosed herein are plasmids of any preceding aspect wherein the plasmid is for use with clustered regularly interspaced short palindromic repeat (CRISPR)/CRISPR-associated integration systems, such as CRISPR/Cas9 integration systems, and further comprises in order a left homology arm, a transgene polynucleotide sequence encoding the CAR, and a right homology arm; wherein the left and right homology arms are each 1000 bp in length or less. In one aspect, the plasmid further comprises a polyadenylation signal between the transgene encoding the CAR and the right homology arm. In some aspects, the left homology arm and right homology arm are different lengths. In other aspects, the left homology arm and right homology arm are the same length.

Also disclosed herein are plasmids of any preceding aspect, wherein the homology arms are 30 bp, 300 bp, 600 bp, or 1000 bp in length.

In one aspect, disclosed herein are plasmids of any preceding aspect, wherein the homology arms specifically hybridize to the suppressor of cytokine signaling 3 (SOCS3) locus.

Also disclosed herein are Adeno-associated viral (AAV) vectors (such as, for example an AAV6 vector) comprising the plasmid of any preceding aspect. In some aspects, the vector further comprises a plasmid encoding a crRNA, tracer RNA (tracrRNA), and a CAS endonuclease.

Also disclosed herein are AAV vectors of any preceding aspect, wherein the vector is a single stranded AAV (ssAAV) or a self-complimentary AAV (scAAV).

In one aspect, disclosed herein are engineered cells (such as, for example engineered NK or NK T cells) comprising the CAR of any of any preceding aspect, the plasmid of any preceding aspect, or the AAV vector of any preceding aspect. For example, in one aspect, disclosed herein are engineered immune cells (such as, for example engineered NK or NK T cells) comprising a transgene encoding chimeric antigen receptor (CAR) comprising an endodomain comprising a STAT3 phosphorylation site (such as, for example, an IL-6, IFN-λR1, IL-2R, IL-10, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the transgene is inserted into the suppressor of cytokine signaling 3 (SOCS3) locus. In one aspect, the CAR of the engineered cell further comprises a co-stimulatory domain (such as, for example, a 2B4 domain, a CD28 co-stimulatory domain, a CD32 domain, a 4-1BB co-stimulatory domain, or any combination thereof).

Also disclosed herein are methods of treating, inhibiting, reducing, decreasing, ameliorating, and/or preventing a cancer and/or metastasis (such as, for example, leukemia (including, but not limited to acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject the engineered cell of any preceding aspect. For example, disclosed herein are methods of treating, inhibiting, reducing, decreasing, ameliorating, and/or preventing a cancer and/or metastasis (such as, for example, leukemia (including, but not limited to acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject engineered immune cell such as, for example engineered NK or NK T cells) comprising a transgene encoding chimeric antigen receptor (CAR) comprising an endodomain comprising a STAT3 phosphorylation site (such as, for example, an IL-6, IL-10, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the transgene is inserted into the suppressor of cytokine signaling 3 (SOCS3) locus. In one aspect, the CAR of the engineered cell used in the disclosed methods further comprises a co-stimulatory domain (such as, for example, a 2B4 domain, a CD28 co-stimulatory domain, a CD32 domain, a 4-1BB co-stimulatory domain, or any combination thereof).

In one aspect, disclosed herein are methods of increasing proliferation of a NK cell and/or NK T cell at the site of an immune response comprising a) obtaining a ribonucleoprotein (RNP) complex comprising a CRISPR/Cas endonuclease (such as Cas X or a class 2 CRISPR/Cas endonuclease, such as Cas9) complexed with a corresponding CRISPR/Cas guide RNA or a nucleic acid encoding the RNP complex and an AAV vector (such as, for example AAV6) comprising a plasmid comprising a transgene polynucleotide sequence encoding a chimeric antigen receptor (CAR) polypeptide; wherein the polynucleotide sequence is flanked by homology arms; wherein the homology arms are 800 bp in length or less; wherein the CAR comprises an endodomain comprising a STAT3 phosphorylation site (such as, for example, an IL-6, IL-10, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); b) introducing the polynucleotide sequence and the RNP complex or a nucleic acid encoding the RNP complex into the NK cell or NK T cell (such as, for example, by electroporation or transfection); wherein the polynucleotide sequence is introduced into the NK cell or NK T cell via infection with the AAV into the NK cell or NK T cell (including infection at multiplicity of infection (MOI) of about 5×10³ to about 5×10⁵); wherein the RNP complex hybridizes to a suppressor of cytokine signaling 3 (SOCS3) locus within the genomic DNA of the NK cell or NK T cell and the NK cell's or NK T cell's DNA repair enzymes insert the transgene encoding CAR into the host genome at the SOCS3 locus within the genomic DNA of the NK cell or NK T cell, thereby creating an engineered NK cell or engineered NK T cell; and administering the engineered NK cell or engineered NK T cell to a subject wherein binding of the CAR to the receptor on a target cell causes phosphorylation through the endodomain of the engineered NK cell or engineered NK T cell and production of more CAR on the surface of the cell and proliferation of the engineered NK cell or engineered NK T cell. In some aspects, the CAR further comprises a co-stimulatory domain (such as, for example, a 2B4 domain, a CD28 co-stimulatory domain, a CD32 domain, a 4-1BB co-stimulatory domain, or any combination thereof).

Also disclosed herein are methods of increasing NK cell or NK T cell proliferation of any preceding aspect, wherein the RNP complex is encoded on the same or a different AAV. In some aspects, disclosed herein are methods of increasing NK cell or NK T cell proliferation of any preceding aspect, wherein the left homology arm and right homology arm are different lengths. In other aspects, the left homology arm and right homology arm are the same length.

Also disclosed herein are methods of increasing NK cell or NK T cell proliferation of any preceding aspect, wherein the homology arms are 30 bp, 300 bp, 600 bp, or 1000 bp in length.

In some aspects, disclosed herein are methods of increasing NK cell or NK T cell proliferation of any preceding aspect, wherein the vector is a single stranded AAV (ssAAV) or a self-complimentary AAV (scAAV).

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 is a cartoon representation of a resting state NK cell with self-driving (self-regulating) CAR inserted into the SOCS3 locus, having low-level baseline expression. A Cas/ribonucleoprotein complex (Cas9) targets the SOCS3 locus, which contains a STAT3 binding site, enabling integration of the CAR transgene delivered by an AAV6 vector at the SOCS3 locus. In the resting state, STAT3 remains unphosphorylated and, therefore, STAT3-activated gene transcription does not occur at the SOCS3 locus, meaning minimal CAR expression.

FIG. 2 is a cartoon representation of an activated-state NK cell in which binding of the target to the self-driving (self-regulating) CAR initiates STAT3 phosphorylation, which drives expression of the SOCS3 locus, resulting in increased CAR expression. A Cas9/ribonucleoprotein complex targets the SOCS3 locus, which contains a STAT3 binding site, enabling integration of the CAR transgene delivered by an AAV6 vector at the SOCS3 locus. Binding of the membrane bound CAR to its target antigen (e.g., cancer antigen) initiates cytokine-mediated signaling at the IL-21 endodomain of the CAR and activates the JAK/STAT pathway to mediate phosphorylation of tyrosine residues. In turn, STAT3 proteins undergo phosphorylation to form STAT dimers, which translocate from the cytoplasm to the nucleus where they bind the SOCS3 locus and activate transcription of the CAR transgene. In the activated state, expression of the CAR transgene facilitates production of and expression of the CAR on the cell surface. Thus, binding of the CAR to its target antigen drives additional CAR expression, effectively creating a self-regulating or self-driving CAR.

FIG. 3 shows the overall structure of an AAV packaging plasmid for site-directed insertion of CAR into AAVS1 locus through 300 bp left and right homology arms.

FIG. 4 shows restriction mapping of a CAR transgene bearing an IL-6R-YMPQ site for STAT3 signaling.

FIG. 5 shows restriction mapping of a CAR transgene bearing a truncated IL-2R-YMPQ site for STAT3 signaling.

FIG. 6 shows restriction mapping of a CAR transgene bearing an IFNLR1-JAK site for STAT3 signaling.

FIG. 7 is a graph depicting RNA-seq data showing upregulation of SOCS3 expression in naive NK cells in response to STAT3 signaling initiated by exposure to IL21, which is maintained in expanded NK cells. Naïve stimulated NK cells were upregulated by STAT3 signaling by a mean 23-fold upregulation as compared to naive NK cells (p=0.0007). Expanded stimulated NK cells demonstrated a mean 25-fold upregulation by STAT3 signaling as compared to resting expanded NK cells (p<0.0001).

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A DNA sequence that “encodes” a particular RNA is a DNA nucleic acid sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, “transgene” refers to exogenous genetic material (e.g., one or more polynucleotides) that has been or can be artificially provided to a cell. The term can be used to refer to a “recombinant” polynucleotide encoding any of the herein disclosed polypeptides that are the subject of the present disclosure. The term “recombinant” refers to a sequence (e.g., polynucleotide or polypeptide sequence) which does not occur in the cell to be artificially provided with the sequence, or is linked to another polynucleotide in an arrangement which does not occur in the cell to be artificially provided with the sequence. It is understood that “artificial” refers to non-natural occurrence in the host cell and includes manipulation by man, machine, exogenous factors (e.g., enzymes, viruses, etc.), other non-natural manipulations, or combinations thereof. A transgene can comprise a gene operably linked to a promoter (e.g., an open reading frame), although is not limited thereto. Upon artificially providing a transgene to a cell, the transgene may integrate into the host cell chromosome, exist extrachromosomally, or exist in any combination thereof.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Chimeric Antigen Receptors (CARs), Plasmids, Vectors, and Engineered Cells

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular chimeric antigen receptor, plasmid, vector, or engineered NK cell/NK T cell is disclosed and discussed and a number of modifications that can be made to a number of molecules including the chimeric antigen receptor, plasmid, vector, or engineered NK cell/NK T cell are discussed, specifically contemplated is each and every combination and permutation of chimeric antigen receptor, plasmid, vector, or engineered NK cell/NK T cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

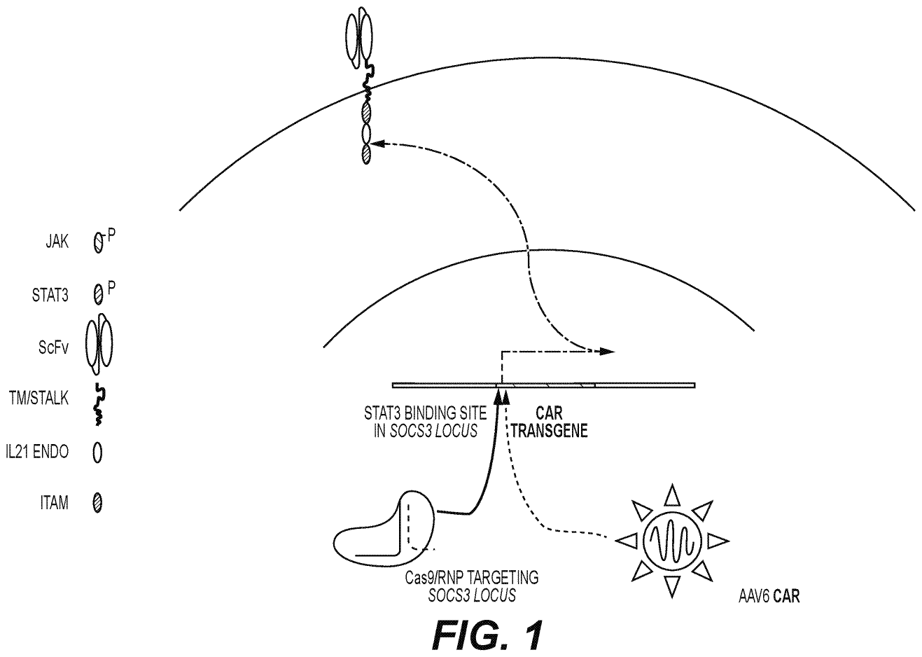

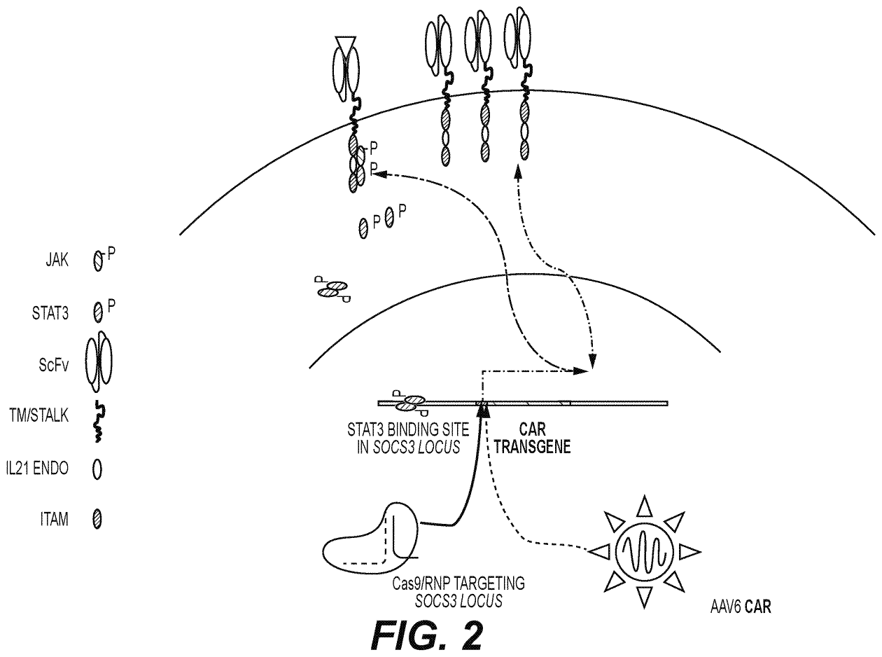

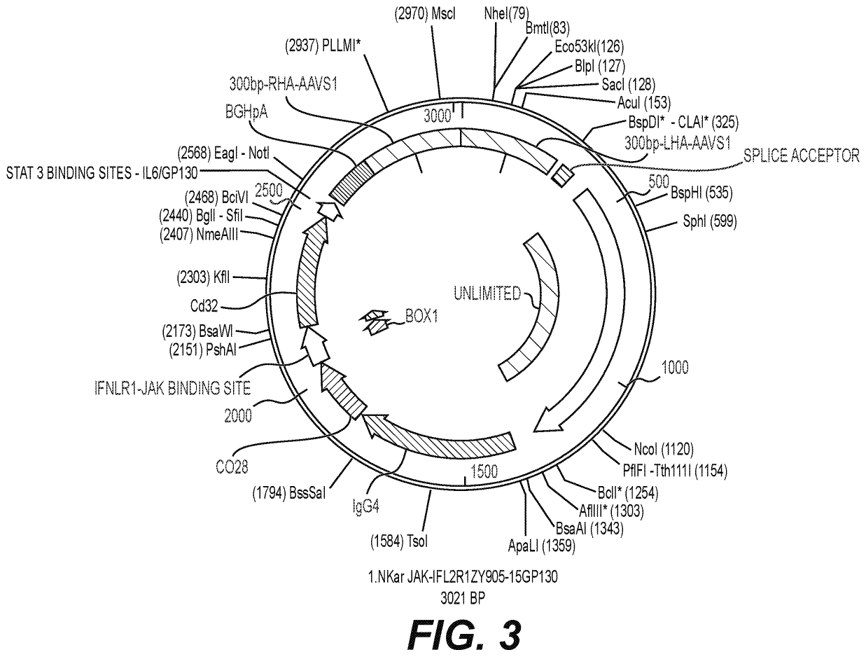

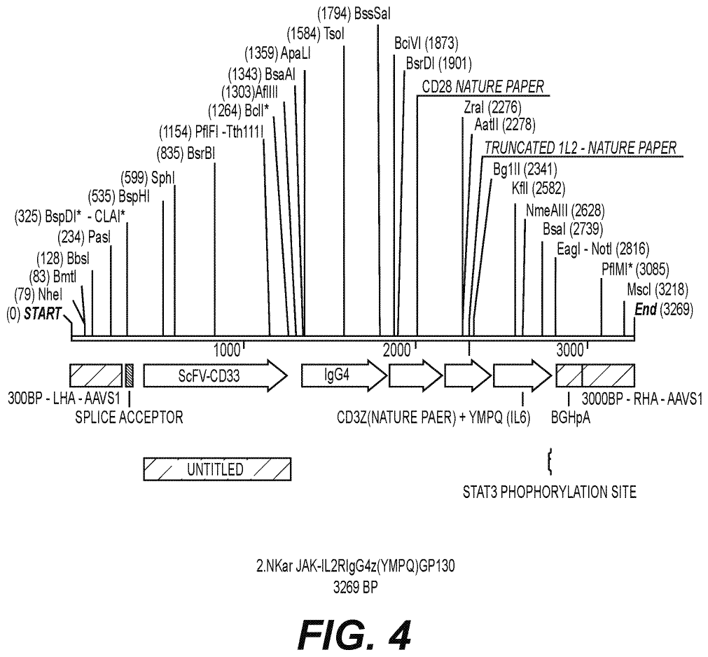

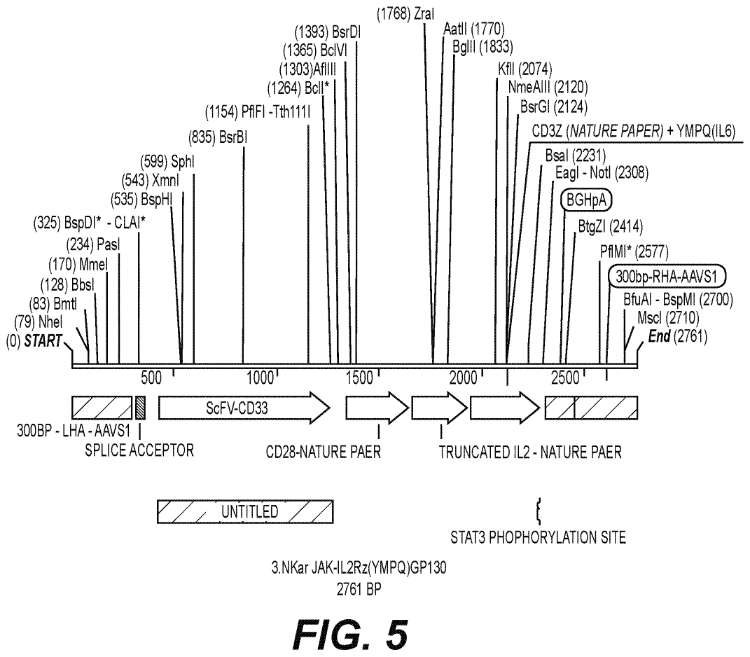

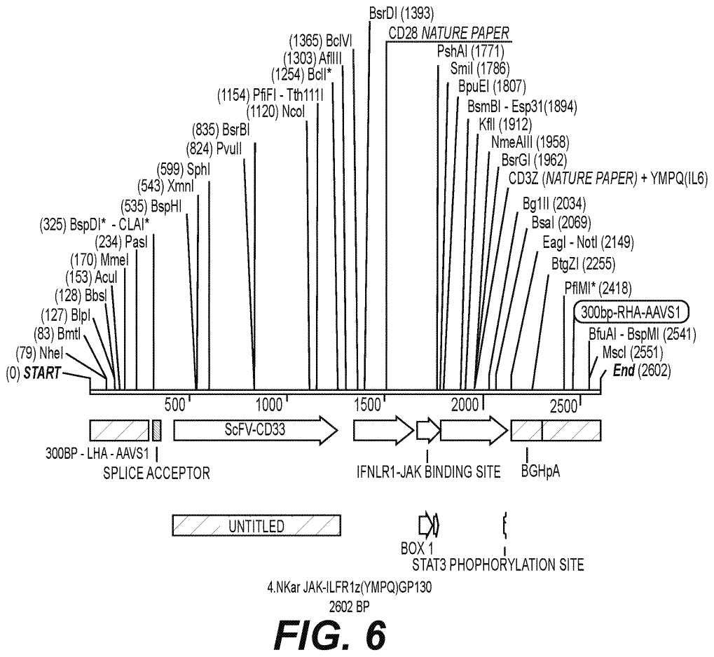

Genetic modification of NK cells to enhance cancer immunotherapy has potential application to treat a wide range of cancers. Disclosed herein is a strategy in which CRISPR/Cas elements, such as CRISPR/Cas9 elements, CRISPR Cas12 elements, or CRISPR/CasX elements, are introduced into NK cells to generate large numbers of genetically modified NK cells. In certain embodiments, the CRISPR/Cas elements comprise a ribonucleoprotein (RNP) complex comprising a CRISPR/Cas endonuclease complexed with a CRISPR/Cas guide RNA. In certain embodiments, the CRISPR/Cas elements comprise a nucleic acid encoding the RNP and the CRISPR/Cas guide RNA. The RNP and the CRISPR/Cas guide RNA may be encoded by a single nucleic acid or separate nucleic acids. This method, using a CRISPR/Cas system, can be used to incorporate a nucleic acid encoding a chimeric antigen receptor and disrupt suppressor of cytokine signaling 3 (SOCS3) by targeting the DNA incorporation to SOCS3. Expression of SOCS3 is typically induced by various cytokines including IL-2, IL-6, IL-10, IFN-γ, and IL-21. The CAR was designed to bind to a target antigen and signal the bound state via the STAT3 phosphorylation site from a number of cytokine receptor endodomains (such as, IL-2, IL-6, IL-10, IFN-γ, and IL-21 endodomains, which typically induce transcription of SOCS3) to induce transcription and expression of the integrated CAR (FIGS. 1 and 2). Thus, binding of the CAR to its target resulted in additional CAR production (FIG. 2). When no target is around to bind the CAR, only a background level of CAR production occurs (FIG. 1). Accordingly, disclosed herein are chimeric antigen receptors (CARs) comprising an endodomain comprising a STAT3 phosphorylation site (such as, for example, an IL-6, IL-10, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33) (see FIGS. 3, 4, 5, and 6).

As used herein “chimeric antigen receptor” or “CAR” refers to a chimeric receptor that targets a cancer antigen and brings the cell expressing the receptor to a cancer cell expressing the target antigen. Typically, the CAR comprises a molecule that recognizes peptides derived from the tumor antigen presented by MHC molecules, or an antibody or fragment thereof (such as for example, a Fab′, scFv, or Fv) expressed on the surface of the CAR cell that targets a cancer antigen. The receptor is fused to a signaling domain (such as, for example, the CD3ζ domain for T cells and NKG2C, NKp44, or CD3ζ domain for NK cells or NK T cells) via a linker. Tumor antigen targets are proteins that are produced by tumor cells that elicit an immune response, particularly NK cell, and NK T cell mediated immune responses. The selection of the antigen binding domain will depend on the particular type of cancer to be treated. Tumor antigens are well known in the art and include, for example, a glioma-associated antigen, carcinoembryonic antigen (CEA), EGFRvIII, IL-IlRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2. NCAM, p53, p53 mutant, Ras mutant, gplOO, prostein, OR51E2, PANX3, PSMA, PSCA, Her2/neu, hTERT, HMWMAA, HAVCR, VEGFR2, PDGFR-beta, survivin and telomerase, legumain, HPV E6, E7, sperm protein 17, SSEA-4, tyrosinase, TARP, WTI, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2. GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. Non-limiting examples of tumor antigens include the following: Differentiation antigens such as tyrosinase. TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, p180erbB-3, c-met, nm-23H1, PSA, IL13Ra2, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG1 6, TA-90\Mac-2 binding protein\cyclophilm C-associated protein, TAAL6, TAG72, TLP, TPS, GPC3, MUC16, LMP1, EBMA-1, BARF-1, CSI, CD319, HER1, B7H6, L1CAM, IL6, and MET.

The CAR polypeptide can also comprise a transmembrane domain (such as, for example, an NKG2D transmembrane domain, a CD28 transmembrane domain, and/or a CD3ξ transmembrane domain) and a co-stimulatory domain (such as, for example, a 2B4 domain, a CD28 co-stimulatory domain, a 4-1 BB co-stimulatory domain, or any combination of a 2B4 domain, a CD28 co-stimulatory domain and/or a 4-1 BB co-stimulatory domain).

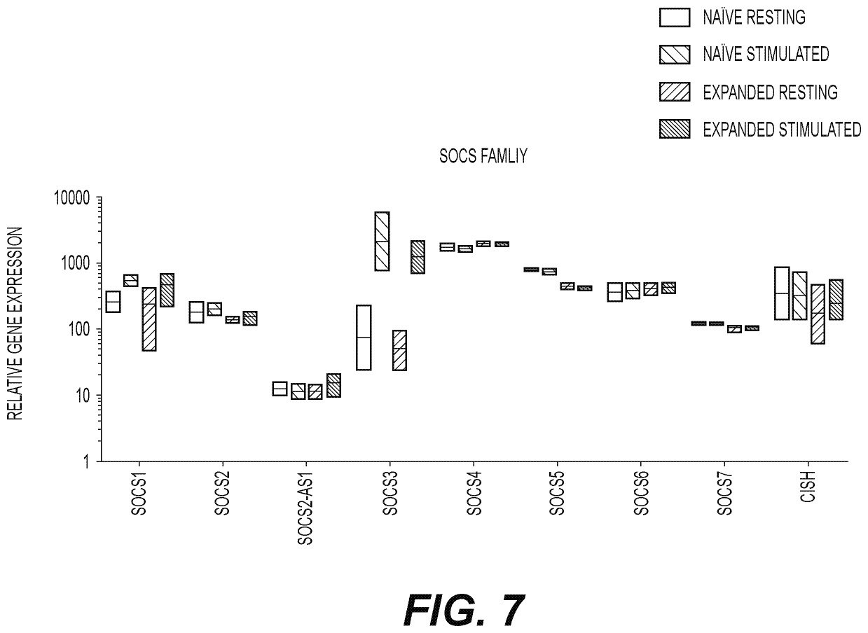

The endodomain of the CAR is what facilitates signaling to the SOCS3 promoter. In normal NK cells and NK T cells, cytokine binding to its cognate cytokine receptor activates the endodomain of the cytokine receptor such as, for example, an IL-10, IL-6, IFN-λR1, IL-2R, or IL-21, leading to the activation of STAT3, which activates gene transcription in the nucleus, including an upregulation of SOCS3 (see FIG. 7). However, by inserting the transgene of the CAR into SOCS3, it is the CAR that is upregulated.

61. Also disclosed herein are plasmids encoding any CAR disclosed herein. For example, disclosed herein are plasmids encoding a CAR comprising an endodomain comprising a STAT3 phosphorylation site (such as, for example, an IL-10, IL-6, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the plasmid is for use with clustered regularly interspaced short palindromic repeat (CRISPR)/CRISPR-associated integration systems, such as a CRISPR/Cas9 integration system, and further comprises in order a left homology arm, a polynucleotide sequence encoding the CAR, and a right homology arm; wherein the left and right homology arms are each 1000 bp in length or less. In one aspect, the plasmid further comprises a polyadenylation signal between the polynucleotide sequence encoding the CAR and the right homology arm.

In general, “CRISPR system” or “CRISPR integration system” refers collectively to transcripts and other elements involved in the expression of or directing the activity of CRISPR-associated “Cas” genes. In some embodiments, the CRISPR/Cas integration system comprises a Class 1, Class 2 or Class 3 CRISPR/Cas system. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

Endonuclease/RNPs (for example, a Cas/RNP) are comprised recombinant endonuclease protein (for example, a Cas9, Cas12a, or CasX endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of an RNA that can hybridize to a target sequence complexed with a complementary repeat RNA (crRNA) and trans complementary repeat RNA (tracrRNA). Accordingly, the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. In some cases, the CRISPR/Cas endonuclease is a CasX polypeptide and the corresponding CRISPR/Cas guide RNA is a CasX guide RNA. These Cas/RNPs are capable of cleaving genomic targets with higher efficiency as compared to foreign DNA-dependent approaches due to their delivery as functional complexes. Additionally, rapid clearance of Cas/RNPs from the cells can reduce the off-target effects such as induction of apoptosis.

To make the RNP complex, crRNA and tracrRNA can be mixed at a 1:1, 2:1, or 1:2 ratio of concentrations between about 50 μM and about 500 μM (for example, 50 μM, 60 μM, 70 μM, 80 μM, 90 μM, 100 μM, 125 μM, 150 μM, 175 μM, 200 μM, 225 μM, 250 μM, 275 μM. 300 μM, 325 μM, 350 μM, 375 μM, 400 μM, 425 μM, 450 μM, 475 μM, or 500 μM), preferably between 100 μM and about 300 μM, most preferably about 200 μM at 95° C. for about 5 min to form a crRNA: tracrRNA complex (i.e., the guide RNA). The crRNA: tracrRNA complex can then be mixed with between about 20 μM and about 50 μM (for example 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM. 39 μM, 40 μM, 41 μM, 42 μM, 43 μM, 44 μM, 45 μM, 46 μM, 47 μM, 48 μM, 49 μM, or 50 μM) final dilution of a Cas endonuclease (such as, for example, Cas9).

Once bound to the target sequence in the target cell, the CRISPR loci can modify the genome by introducing into the target DNA insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof. Thus, the disclosed methods can be used to generate knock-outs, or knock-ins when combined with DNA for homologous recombination. It is shown herein that transduction via Adeno-associated viral (AAV) of Cas/RNPs is an efficient method that overcomes the previous constraints of genetic modification in NK cells and NK T cells.

The maximum AAV packaging capacity of about 4.5 kilobases limits the donor size which includes homology arms. Accordingly, in certain embodiments, any transcript above 100 bp and any transgene may have homology arms that are at least 800 bp for each arm with many systems employing asymmetric arms of 800 bp and 1000 bp for a total of 1800 bp. Thus, the AAV vector cannot deliver a transgene larger than about 2.5 kb. In one aspect, disclosed herein are AAV CRISPR/CAS9 nucleotide delivery systems comprising a donor construct plasmid with homology arms between 30 bp and 1000 bp, including, but not limited to 30 bp, 50 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp. 930 bp, 940 bp, 950 bp, 960 bp, 970 bp, 980 bp, 990 bp, or 1000 bp. For example, the homology arms can be symmetrical 30 bp homology arms, symmetrical 300 bp homology arms, symmetrical 500 bp homology arms, symmetrical 600 bp homology arms, symmetrical 800 bp homology arms, symmetrical 1000 bp homology arms, or asymmetrical 800 bp homology arms comprising a 800 bp left homology arm (LHA) and a 1000 bp right homology arm (RHA) for homologous recombination (HR) or no homology arms at all for non-homologous end joining using homology-independent targeted integration (HITI) plasmids.

It is understood and herein contemplated that homology arms can be symmetrical (same length on each side) or asymmetrical (different lengths on each side) to accommodate differing transgene lengths. That is, homology arm lengths can have any combination of left homology arm (LHA) length and right homology arm (RHA) length including but not limited to LHA 30 bp and RHA 30 bp, LHA 30 bp and RHA 100 bp, LHA 30 bp and RHA 300 bp, LHA 30 bp and RHA 500 bp, LHA 30 bp and RHA 800 bp, LHA 30 bp and RHA 1000 bp, LHA 100 bp and RHA 30 bp, LHA 100 bp and RHA 100 bp, LHA 100 bp and RHA 300 bp, LHA 100 bp and RHA 500 bp, LHA 100 bp and RHA 800 bp, LHA 100 bp and RHA 1000 bp, LHA 300 bp and RHA 30 bp, LHA 300 bp and RHA 100 bp, LHA 300 bp and RHA 300 bp, LHA 300 bp and RHA 500 bp, LHA 300 bp and RHA 800 bp, LHA 300 bp and RHA 1000 bp, LHA 500 bp and RHA 30 bp, LHA 500 bp and RHA 100 bp, LHA 500 bp and RHA 300 bp, LHA 500 bp and RHA 500 bp, LHA 500 bp and RHA 800 bp, LHA 500 bp and RHA 1000 bp, LHA 800 bp and RHA 30 bp, LHA 800 bp and RHA 100 bp, LHA 800 bp and RHA 300 bp, LHA 800 bp and RHA 500 bp, LHA 800 bp and RHA 800 bp, LHA 800 bp and RHA 1000 bp, LHA 1000 bp and RHA 30 bp, LHA 1000 bp and RHA 100 bp, LHA 1000 bp and RHA 300 bp, LHA 1000 bp and RHA 500 bp, LHA 1000 bp and RHA 800 bp, and LHA 1000 bp and RHA 1000 bp.

There are several ways to provide the DNA template, including viral and non-viral methods. In non-viral approaches, the single-stranded or double-stranded DNA template is typically electroporated along with Cas/RNP, however, it has a lower efficiency in comparison to viral transduction. For viral gene delivery, adeno-associated viruses (AAV), including AAV6, were used safely in clinical trials and are useful as vectors for sensitive primary immune cells, including T-cells. It is also possible replace the Cas/RNP with a nucleic acid encoding the Cas/RNP.

In some aspects, the homology arms specifically hybridize to the suppressor of cytokine signaling 3 (SOCS3) locus.

Also disclosed herein are plasmids that can be integrated into the genome of the transduced cells via HITI, CRISPaint, or other nonhomologous end joining (NHEJ). As such, they have an advantage of integrating with higher efficiency. In some examples, the plasmids for NHEJ are those disclosed in International Publication Number WO2020/198675, which is incorporated herein by reference in its entirety. To aid in the identification of cleavage site to remove the transgene for integration, the plasmids may comprise the protospacer adjacent motif (PAM) and crRNA (i.e., the gRNA) to target the donor transgene integration. In some examples, for the NHEJ DNA templates (e.g., CRISPaint DNA templates), a single (PAMg) or a double (PAMgPAMg) Cas-targeting sequence (e.g., Cas9, Cas12, or CasX-targeting sequence) is incorporated around the transgene (e.g., a polynucleotide encoding the CAR, such as CD33 CAR, disclosed herein) but within the inverted terminal repeats (ITRs). Therefore, Cas can simultaneously cut gDNA and the CRISPaint DNA template, enabling integration at the genomic double-stranded break.

As noted above, the use of the AAV as a vector to deliver the disclosed CRISPR/Cas9 plasmid and any donor transgene is limited to a maximum of ˜4.5 kb. It is understood and herein contemplated that one method of increasing the allowable size of the transgene is to create additional room by exchanging the Cas9 of Streptococcus pyogenes (SpCas9) typically used for a synthetic Cas9, or Cas9 from a different bacterial source. Substitution of the Cas9 can also be used to increase the targeting specificity so less gRNA needs to be used. Thus, for example, the Cas9 can be derived from Staphylococcus aureus (SaCas9), Acidaminococcus sp. (AsCpf1), Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fokl-Fused dCas9, expanded Cas9 (xCas9), and/or catalytically dead Cas9 (dCas9).

It is understood and herein contemplated that the disclosed plasmids and vectors can be incorporated into a cell (for example, a NK cell or NK T cell) for use in adoptive immunotherapy where the NK cell or NK T cell having incorporated the plasmid and/or vector expresses a CAR. Thus, disclosed herein are engineered cells (such as, for example engineered NK or NK T cells) comprising any of the CARs disclosed herein, any of the plasmids disclosed herein, and/or any of the AAV vectors disclosed herein. For example, in one aspect, disclosed herein are engineered immune cells (such as, for example engineered NK or NK T cells) comprising a transgene encoding chimeric antigen receptor (CAR) comprising an endodomain comprising a STAT3 phosphorylation site (such as, for example, an IL-10, IL-6, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the transgene is inserted into the suppressor of cytokine signaling 3 (SOCS3) locus. In one aspect, the CAR of the engineered cell further comprises a co-stimulatory domain (such as, for example, a 2B4 domain, a CD28 co-stimulatory domain, a CD32 domain, a 4-1BB co-stimulatory domain, or any combination thereof).

Prior to the transduction of the cells (such as, for example, NK cells and/or NK T cells), the cell can be incubated in a media suitable for the propagation of the cells. It is understood and herein contemplated that the culturing conditions can comprise the addition of cytokines, antibodies, and/or feeder cells. Thus, in one aspect, disclosed herein are methods of genetically modifying a cell (such as, for example, NK cells and/or NK T cells), further comprising incubating the cells for at least 1, 2, 3, 4, 5, 6, 7, 8 9, 10, 11, 12, 13, or 14 days prior to transducing the cells in media that supports the propagation of cells; wherein the media further comprises cytokines, antibodies, and/or feeder cells. For example, the media can comprise IL-2, IL-12, IL-15, IL-18, and/or IL-21. In one aspect, the media can also comprise anti-CD3 antibody. In one aspect, the feeder cells can be purified from feeder cells that stimulate cells. For example, NK cell stimulating feeder cells for use in embodiments disclosed herein, can be either irradiated autologous or allogeneic peripheral blood mononuclear cells (PBMCs) or nonirradiated autologous or PBMCs; RPMI8866; HFWT, K562; K562 cells transfected with membrane bound IL-15 and 4-1BBL, or membrane bound IL-21 and 4-1BBL; plasma membrane vesicles comprising membrane bound IL-15 and 4-1BBL, or membrane bound IL-21 and 4-1BBL, such as plasma membrane vesicles obtained from K562 cells; or any combination thereof; or EBV-LCL. In some aspects, the feeder cells may be provided in combination with a solution of IL-21, IL-15, and/or 4-1BBL. Feeder cells can be seeded in the culture of cells at a 1:2, 1:1, or 2:1 ratio. It is understood and herein contemplated that the period of culturing can be between 1 and 14 days post AAV infection (i.e., 1, 2, 3, 4, 5, 6, 7, 8, 9, 10, 11, 12, 13, or 14 days), preferably between 3 and 7 days, most preferably between 4 and 6 days.

It is understood and herein contemplated that the incubation conditions for primary cells and expanded cells (including, but not limited to primary and expanded NK cells and/or NK T cells) can be different. In one aspect, the culturing of primary NK cells or NK T cells prior to AAV infection comprises media and cytokines (such as, for example, IL-2, IL-12, IL-15, IL-18, and/or IL-21) and/or anti-CD3 antibody for less than 5 days (for example 1, 2, 3, or 4 days). For expanded NK cells the culturing can occur in the presence of NK feeder cells (at for example, a 1:1 ratio) in addition to or in lieu of cytokines (such as, for example, IL-2, IL-12, IL-15, IL-18, and/or IL-21) and/or anti-CD3 antibody. Culturing of expanded NK cells can occur for 1, 2, 3, 4, 5, 6, 7, 8, 9, or 10 days prior to transduction. Thus, in one aspect, disclosed herein are methods of genetically modifying a cell (such as for example, a T cell, B cell, macrophage, NK cell, NK T cells, fibroblast, neuronal cell osteoblast, hepatocyte, epithelial cell, and/or muscle cell) comprising incubating primary cells for 4 days in the presence of IL-2 prior to infection with an AAV vector and/or electroporation (when the RNP complex is introduced via electroporation) or incubating expanded cells in the presence of irradiated feeder cells for 4, 5, 6, or 7 days prior to infection with AAV and/or electroporation when the RNP complex is introduced via electroporation.

Following transduction (e.g., via AAV infection or electroporation) of the cell (such as, for example, a NK cell and/or NK T cell), the now engineered cell can be propagated in a media comprising feeder cells that stimulate the modified cells (such as, for example, a NK cell and/or NK T cell). Thus, the engineered cells retain viability and proliferative potential, as they are able to be expanded post-AAV infection and/or electroporation (when the RNP complex or nucleic acid encoding the RNP is introduced, e.g., via electroporation) using irradiated feeder cells. For example, NK cell stimulating feeder cells for use in embodiments disclosed herein can be either irradiated autologous or allogeneic peripheral blood mononuclear cells (PBMCs) or nonirradiated autologous or PBMCs; RPMI8866; HFWT. K562; K562 cells transfected with membrane bound IL-15 and 41BBL, or membrane bound IL-21 and 4-1BBL; plasma membrane vesicles comprising membrane bound IL-15 and 4-1BBL, or membrane bound IL-21 and 4-1BBL, such as plasma membrane vesicles obtained from K562 cells; or any combination thereof; or EBV-LCL. In some aspects, the NK cell feeder cells may be provided in combination with a solution of IL-21, IL-15, and/or 41BBL. Feeder cells can be seeded in the culture of NK cells at a 1:2, 1:1, or 2:1 ratio. It is understood and herein contemplated that the period of culturing can be between 1 and 14 days post infection and/or electroporation (i.e., 1, 2, 3, 4, 5, 6, 7, 8, 9, 10, 11, 12, 13, or 14 days), preferably between 3 and 7 days, most preferably between 4 and 6 days. In some aspects, the media for culturing the modified NK cells can further comprise cytokines such as, for example, IL-2, IL-12, IL-15, IL-18, and/or IL-21.

In one aspect, it is understood and herein contemplated that expression of the disclosed CARs not only creates a feedback loop for CAR expression, but also increases proliferation of the NK cells and/or NK T cells, while also inhibiting regulator suppression of NK cells and NK T cells by SOCS3. In one aspect, disclosed herein are methods of increasing proliferation of a NK cell and/or NK T cell at the site of an immune response comprising a) obtaining a ribonucleoprotein (RNP) complex comprising a CRISPR/Cas endonuclease (e.g., CasX or a class 2 CRISPR/Cas endonuclease, such as Cas9) complexed with a corresponding CRISPR/Cas guide RNA or a nucleic acid encoding the RNP complex and an AAV vector (such as, for example AAV6) comprising a plasmid comprising a polynucleotide sequence encoding a chimeric antigen receptor (CAR) polypeptide; wherein the polynucleotide sequence is flanked by homology arms; wherein the homology arms are 800 bp in length or less; wherein the CAR comprises an endodomain comprising a STAT3 phosphorylation site (such as, for example, an IL-10, IL-6, IFN-λR1, IL-2R, or IL-21 endodomain), a transmembrane domain such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); b) introducing the polynucleotide sequence and the RNP complex or a nucleic acid encoding the RNP complex into the NK cell or NK T cell (such as, for example, by electroporation or transfection); wherein the polynucleotide sequence is introduced into the NK cell or NK T cell via infection with the AAV into the NK cell or NK T cell (including infection at multiplicity of infection (MOI) of about 5×10³ to about 5×10⁵); wherein the RNP complex hybridizes to a suppressor of cytokine signaling 3 (SOCS3) locus within the genomic DNA of the NK cell or NK T cell and the NK cell's or NK T cell's DNA repair enzymes insert the transgene into the host genome at the SOCS3 locus within the genomic DNA of the NK cell or NK T cell thereby creating an engineered NK cell or engineered NK T cell; and administering the engineered NK cell or engineered NK T cell to a subject wherein binding of the CAR to the receptor on a target cell causes phosphorylation through the endodomain of the engineered NK cell or engineered NK T cell and production of more CAR on the surface of the cell and proliferation of the engineered NK cell or engineered NK T cell. In some aspects, the CAR further comprises a co-stimulatory domain (such as, for example, a 2B4 domain, a CD28 co-stimulatory domain, a CD32 domain, a 4-1BB co-stimulatory domain, or any combination thereof).

### 1. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with Cor A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the limiting nucleic acid is bound to the non-limiting nucleic acid. Typically, the non-limiting primer is in for example, 10 or 100 or 1000-fold excess. This type of assay can be performed under conditions where both the limiting and non-limiting primer are for example, 10-fold or 100-fold or 1000-fold below their k_{d}, or where only one of the nucleic acid molecules is 10-fold or 100-fold or 1000-fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer is enzymatically manipulated under conditions which promote the enzymatic manipulation. For example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 2. Nucleic Acids

There are a variety of molecules disclosed herein that are nucleic acid based. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C. G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Sequences

There are a variety of sequences related to the protein molecules involved in the signaling pathways disclosed herein, for example CD33, 4-1BB, NKG2D, or 2B4, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

### c) Primers and Probes

Disclosed are compositions including primers and probes, which are capable of interacting with the disclosed nucleic acids, such as the CD33 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the CD33 gene typically will be used to produce an amplified DNA product that contains a region of CD33 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, can be delivered in aerosol formulation, and can transfect non-dividing cells. Pox viral vectors are large and have several sites for inserting genes, and they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. AAVs used can be derived from any AAV serotype, including but not limited to AAC1, AAV2, AAV3, AAV4, AAV5. AAV6, AAV7, AAV8, AAV9, and recombinant (rAAV) such as, for example AAV-Rh74, and/or synthetic AAV (such as, for example AAV-DJ, Anc80). AAV serotypes can be selected based on cell or tissue tropism. AAV vectors for use in the disclosed compositions and methods can be single stranded (SS) or self-complementary (SC).

Transcripts that are delivered via AAV vectors can be packaged as a linear single-stranded (ss) DNA with a length of approximately 4.7 kb (ssAAV) or as linear self-complementary (sc) DNA (scAAV). The benefit of the scAAV vector is that it contains a mutated inverted terminal repeat (ITR), which is required for replication and helps to bypass rate-limiting steps of second strand generation in comparison to ssDNA vectors. Due to the limitation in the packaging capacity of scAAV, 30 bp, 300 bp, 500 bp, and 800-1000 bps of homology arms (HAs) for the right and left side of the Cas9-targeting site were designed to find the most optimal length of HAs and to provide possible lengths of HAs to be chosen based on the size of transgenes by researchers. Additionally, due to limitations in packaging capacity compared to ssAAV. scAAV may not be suitable for larger transgenes such as chimeric antigen receptor (CAR) targeting CD33. Therefore, based on the size of transgenes, both ssAAV and scAAV were designed and tested, which provides a wide range of options for gene insertion in primary NK cells and/or NK T cells.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression.

The disclosed vectors thus provide DNA molecules which are capable of integration into a mammalian chromosome without substantial toxicity.

The inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

It is understood and herein contemplated that the packaging capacity of an AAV is limited. One method to overcome the loading capacity of an AAV vector is through the use of two vectors, wherein the transgene is split between the two plasmids and a 3′ splice donor and 5′ splice acceptor are used to join the two sections of transgene into a single full-length transgene. Alternatively, the two transgenes can be made with substantial overlap and homologous recombination will join the two segments into a full-length transcript.

In some embodiments, the method disclosed herein comprises infecting the NK cell with a range of MOI of AAV from about 1 to about 1000K MOI (e.g., about 5 to 500K MOI) of AAV. For example, the method disclosed herein comprising infecting the NK cell with at least about 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 100, 150, 200, 250, 300, 350, 400, 450, or 500 MOI of AAV.

### 4. Expression Systems

The nucleic acids that are delivered to cells typically contain expression controlling systems. For example, the inserted genes in viral and retroviral systems usually contain promoters and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 5. Peptides

### a) Protein Variants

Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | Alle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group. 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln. EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Method of Treating Cancer

Cancer immunotherapy has been advanced in recent years; genetically-modified chimeric antigen receptor (CAR) T cells are an excellent example of engineered immune cells successfully deployed in cancer immunotherapy. These cells were recently approved by the FDA for treatment against CD19+B cell malignancies, but success has so far been limited to diseases bearing a few targetable antigens, and targeting such limited antigenic repertoires is prone to failure by immune escape. Furthermore, CAR T cells have been focused on the use of autologous T cells because of the risk of graft-versus-host disease caused by allogeneic T cells. In contrast, Natural Killer (NK) cells are able to kill tumor targets in an antigen-independent manner and do not cause GvHD, which makes them a good candidate for cancer immunotherapy. It is understood and herein contemplated that the disclosed plasmids and methods can be used to generate, for example, chimeric antigen receptor Natural Killer (CAR NK) T cells and chimeric antigen receptor Natural Killer (CAR NK) cells to target a cancer. The disclosed plasmids, chimeric antigen receptor Natural Killer (CAR NK) T cells and chimeric antigen receptor Natural Killer (CAR NK) cells can be used to manufacture or prepare a medicament for treating a cancer and/or a metastasis. For example, an aspect of the disclosure comprises the use of engineered immune cells such as, for example, engineered NK or NK T cells, in the preparation or manufacture of a medicament for treating cancer, the engineered immune cells comprising a transgene encoding chimeric antigen receptor (CAR) comprising an endodomain comprising a STAT3 phosphorylation site (such as, for example, an IL-10, IL-6, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the transgene is inserted into the suppressor of cytokine signaling 3 (SOCS3) locus. In one aspect, the CAR of the engineered cell used in the disclosed methods further comprises a co-stimulatory domain (such as, for example, a 2B4 domain, a CD28 co-stimulatory domain, a CD32 domain, a 4-1BB co-stimulatory domain, or any combination thereof).

Likewise, disclosed herein are methods of treating, decreasing, reducing, inhibiting, ameliorating, and/or preventing a cancer and/or metastasis (such as, for example, acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to a subject with a cancer any modified cell (for example, engineered NK cells and NK T cells) disclosed herein. Accordingly, in one aspect, disclosed herein are methods of treating, inhibiting, reducing, decreasing, ameliorating, and/or preventing a cancer and/or metastasis (such as, for example, leukemia (including, but not limited to acute lymphocytic leukemia (ALL), acute myeloid leukemia (AML), chronic myeloid leukemia (CML), hairy cell leukemia (HCL), and/or myelodysplastic syndromes (MDS)) in a subject comprising administering to the subject a therapeutically effective amount of an engineered immune cell such as, for example engineered NK or NK T cells) comprising a transgene encoding chimeric antigen receptor (CAR) comprising an endodomain comprising a STAT3 phosphorylation site (such as, for example, an IL-10, IL-6, IFN-λR1, IL-2R, or IL-21 endodomain), a transmembrane domain (such as, for example, a CD28 transmembrane domain, a CD3ζ transmembrane domain, or an NKG2D transmembrane domain), and a single-chain variable fragment (scFV) that specifically binds to a receptor on a target cell (such as, for example CD33); wherein the transgene is inserted into the suppressor of cytokine signaling 3 (SOCS3) locus. In one aspect, the CAR of the engineered cell used in the disclosed methods further comprises a co-stimulatory domain (such as, for example, a 2B4 domain, a CD28 co-stimulatory domain, a CD32 domain, a 4-1BB co-stimulatory domain, or any combination thereof).

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As noted above, the plasmids, vectors, and engineered NK cells and NK T cells disclosed herein can be used to treat, inhibit, reduce, decrease, ameliorate, and/or prevent cancer. A representative but non-limiting list of cancers that the disclosed compositions can be used to treat is the following: lymphoma, B cell lymphoma, T cell lymphoma, mycosis fungoides, Hodgkin's Disease, acute lymphocytic leukemia (ALL), hairy cell leukemia (HCL), myelodysplastic syndromes (MDS), myeloid leukemia (including, but not limited to acute myeloid leukemia (AML) and chronic myeloid leukemia (CML)), bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, the engineered cells cell (e.g., NK cells and/or NK T cells) used in the disclosed immunotherapy methods can be primary cells from a donor source (such as, for example, an allogeneic donor source for an adoptive transfer therapy or an autologous donor source (i.e., the ultimate recipient of the modified cells), a cell line (including, but not limited to NK cell lines NK RPMI8866; HFWT, K562, and EBV-LCL), or from a source of expanded cells derived a primary cell source or cell line. Because primary cells can be used, it is understood and herein contemplated that the disclosed modifications of the cell can occur ex vivo or in vitro.

It is understood and herein contemplated that though disclosed methods of treatment provide a complete and effective way to treat cancer, a comprehensive treatment regimen can include the administration of additional anti-cancer agents. Thus, the disclosed treatments methods can also include the administration any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod). Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine. Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate). JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib. Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab). Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate). Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Treatment methods can include or further include checkpoint inhibitors including, but not limited to, antibodies that block PD-1 (Pembrolizumab, Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

1. A chimeric antigen receptor (CAR) comprising an endodomain comprising a STAT3 phosphorylation site, a transmembrane domain, and a single-chain variable fragment (scFV) that specifically binds to a receptor on a target cell.

2. The CAR of claim 1, wherein the endodomain comprises an IL-10, IL-6, IFN-IR1, IL-2R, or IL-21 endodomain.

3. The CAR of claim 1, wherein the transmembrane domain of the CAR comprises a CD28 transmembrane domain, a CD3ζ transmembrane domain, or an NKG2D transmembrane domain.

4. The CAR of claim 1, wherein the CAR further comprises a co-stimulatory domain selected from a 2B4 domain, a CD28 co-stimulatory domain, a CD32 domain, a 4-1BB co-stimulatory domain, or any combination thereof.

5.-6. (canceled)

7. A plasmid encoding the CAR of claim 1.

8. The plasmid of claim 7, wherein the plasmid is for use with clustered regularly interspaced short palindromic repeat (CRISPR)/CRISPR-associated (Cas) integration systems and further comprises in order a left homology arm, a polynucleotide sequence encoding the CAR, and a right homology arm; wherein the left and right homology arms are each 1000 bp in length or less, and wherein the homology arms specifically hybridize to the Adeno-Associated Virus Integration Site 1 (AAVS1) of the Suppressor of cytokine signaling 3 (SOCS3) locus.

9.-11. (canceled)

12. The plasmid of claim 7, wherein the left homology arm and right homology arm are different lengths or are the same length.

13. (canceled)

14. The plasmid of claim 7, wherein the homology arms are 30 bp, 300 bp, 600 bp, or 1000 bp in length.

15. An Adeno-associated viral (AAV) vector comprising the plasmid of claim 7.

16.-19. (canceled)

20. An engineered cell comprising the CAR of claim 1, the plasmid of claim 7, or the AAV vector of claim 15.

21.-28. (canceled)

29. A method of treating a cancer in a subject comprising administering to the subject the engineered cell of claim 20.

30.-37. (canceled)

38. A method of increasing NK cell and/or NK T cell proliferation at the site of an immune response in a subject comprising
a) obtaining a ribonucleoprotein (RNP) complex comprising a CRISPR/Cas endonuclease complexed with a corresponding CRISPR/Cas guide RNA or a nucleic acid encoding the RNP complex and an AAV vector comprising a plasmid comprising a polynucleotide sequence encoding a chimeric antigen receptor (CAR) polypeptide; wherein the polynucleotide sequence is flanked by homology arms; wherein the homology arms are 800 bp in length or less; wherein the CAR comprises an endodomain comprising a STAT3 phosphorylation site, a transmembrane domain, and a single-chain variable fragment (scFV) that specifically binds to a receptor on a target cell;
b) introducing the polynucleotide sequence and the RNP complex, or a nucleic acid encoding the RNP complex, into the NK cell or NK T cell; wherein the RNP complex hybridizes to a suppressor of cytokine signaling 3 (SOCS3) locus within the genomic DNA of the NK cell or NK T cell and the NK cell's or NK T cell's DNA repair enzymes insert the polynucleotide sequence into the host genome at the SOCS3 locus within the genomic DNA of the NK cell or NK T cell thereby creating an engineered NK cell or engineered NK T cell; and
c) administering the engineered NK cell or engineered NK T cell to a subject wherein binding of the CAR to the receptor on a target cell causes phosphorylation through the endodomain of the engineered NK cell or engineered NK T cell and production of more CAR on the surface of the cell and proliferation of the engineered NK cell or engineered NK T cell.

39. The method of claim 38, wherein the endodomain comprises an IL-10, IL-6, IFN-1R1, IL-2R, or IL-21 endodomain.

40. The method of claim 38, wherein the transmembrane domain of the CAR comprises a CD28 transmembrane domain, a CD3ζ transmembrane domain, or an NKG2D transmembrane domain.

41. The method of claim 38, wherein the CAR further comprises a co-stimulatory domain selected from a 2B4 domain, a CD28 co-stimulatory domain, a CD32 domain, a 4-1BB co-stimulatory domain, or any combination thereof.

42. (canceled)

43. The method of claim 38, wherein the scFv of the CAR specifically binds CD33.

44. The method of claim 38, wherein the cell is infected at a multiplicity of infection (MOI) of about 5 to about 500,000 of the AAV.

45. The method of claim 38, wherein the RNP complex is introduced into the cell via infection with an AAV, via electroporation, or via transfection; and wherein the RNP complex is encoded on the same AAV or on a different AAV.

46.-49. (canceled)

50. The method of claim 38, wherein the left homology arm and right homology arm are the same length or are different lengths.

51. The method of claim 50, wherein the homology arms are 600 bp in length or less.

52.-54. (canceled)
